(19) United States
(12) Patent Application Publication
(10) Pub. No.: US 2022/0378335 A1
(43) Pub. Date: Dec. 1, 2022

(54) ELECTROSTIMULATION-FREE AND BIOMETRICALLY ENCRYPTABLE NONINVASIVE BIOCHEMICAL SENSING DEVICE AND METHOD

(71) Applicant: THE REGENTS OF THE UNIVERSITY OF CALIFORNIA, Oakland, CA (US)

(72) Inventors: Sam Emaminejad, Los Angeles, CA (US); Shuyu Lin, Los Angeles, CA (US); Bo Wang, Los Angeles, CA (US)

(73) Assignee: THE REGENTS OF THE UNIVERSITY OF CALIFORNIA, Oakland, CA (US)

(21) Appl. No.: 17/771,003

(22) PCT Filed: Oct. 21, 2020
(86) PCT No.: PCT/US2020/056701

§ 371 Date: Apr. 21, 2022

Related U.S. Application Data
(60) Provisional application No. 62/924,401, filed on Oct. 22, 2019.

(51) Int. Cl. A61B 5/1477; A61B 5/145; A61B 5/1172; A61B 5/00; H04L 9/08

(52) U.S. Cl. CPC A61B 5/1477; A61B 5/14517; A61B 5/1172; A61B 5/6826; H04L 9/0866; A61B 2562/125; A61B 5/14546

(57) ABSTRACT

Example implementations also include a method of sensing the presence and quantity of a biochemical by applying a current across a biochemical sensing electrode and a reference electrode, contacting a hydrogel layer to a biological surface, absorbing a biofluid from the biological surface into the hydrogel layer, obtaining, at a processor coupled to the biochemical sensing electrode and the reference electrode, a change in current across the biochemical sensing electrode and the reference electrode, and generating, at the processor, a quantitative biochemical response. Example implementations further include obtaining a biometric encryption key based on the biological surface, and encrypting the quantitative response based on a biometric encryption key. Example implementations further include contacting a fingerprint scanner to the biological surface, and obtaining a fingerprint pattern from the biological surface at the fingerprint scanner, where the biometric encryption key is based on the fingerprint pattern.

### CROSS-REFERENCE TO RELATED PATENT APPLICATIONS

This application claims priority to U.S. Provisional Patent Application Ser. No. 62/924,401, entitled “Non-Invasive and In-situ Encrypted Molecular-Level Information Collection and Authentication System,” filed Oct. 22, 2019, the contents of such application being hereby incorporated by reference in its entirety and for all purposes as if completely and fully set forth herein.

### STATEMENT REGARDING FEDERALLY SPONSORED RESEARCH OR DEVELOPMENT

This invention was made with government support under Grant Number 1722972, awarded by the National Science Foundation. The government has certain rights in the invention.

### TECHNICAL FIELD

The present implementations relate generally to biochemical sensor devices, and more particularly to an electrostimulation-free and biometrically encryptable noninvasive biochemical sensing device and method.

### BACKGROUND

Health monitoring is increasingly desired to perform increasingly accurate health diagnostics and guide improved health outcomes for increasing numbers of users and activity scenarios. In particular, detection of biochemical levels of biofluids secreted by a user can provide significant health data and, in turn, drive significantly improved health outcomes. However, conventional systems may not effectively detect and isolate biochemicals in naturally occurring amounts of secreted biofluids at in vivo sites noninvasively and accurately. In addition, conventional systems may detect and isolate biochemicals without privacy safeguards and security. Thus, a technological solution for an electrostimulation-free and biometrically encryptable noninvasive biochemical sensing is desired.

### SUMMARY

Example implementations include a biochemical sensor device with a substrate, a first base electrode layer disposed on the substrate, and a hydrogel layer disposed over the first base electrode layer. Example implementations further include a biochemical sensor device with a carbon nanotube layer disposed on the first base electrode layer. Example implementations further include a biochemical sensor device with a platinum layer disposed on the carbon nanotube layer. Example implementations further include a biochemical sensor device with a poly-m-phenylenediamine (PPD) layer disposed on the platinum layer. Example implementations further include a biochemical sensor device with a lithium oxide layer disposed on the PPD layer.

Example implementations also include an electronic sensor device with a system processor, and a sensor processor operatively coupled to the system processor, a biochemical sensing electrode operatively coupled to the sensor processor, and a hydrogel layer operatively coupled to the biochemical sensing electrode. Example implementations further include an electronic sensor device with a fingerprint scanner device operatively coupled to the system processor.

Example implementations also include a method of manufacturing a biochemical sensor by dissolving agarose powder in acetate buffer to form a buffer solution, injecting the buffer solution into a hydrogel chamber, and solidifying the buffer solution into a hydrogel layer.

Example implementations also include a method of manufacturing a biochemical sensor by depositing a first base electrode layer on a substrate, depositing a carbon nanotube layer on the first base electrode, and depositing a platinum layer on the carbon nanotube layer. Example implementations further include depositing a poly-m-phenylenediamine (PPD) layer on the platinum layer. Example implementations further include depositing a lactate oxide layer on the PPD layer.

Example implementations also include a method of sensing the presence and quantity of a biochemical by applying a current across a biochemical sensing electrode and a reference electrode, contacting a hydrogel layer to a biological surface, absorbing a biofluid from the biological surface into the hydrogel layer, obtaining, at a processor coupled to the biochemical sensing electrode and the reference electrode, a change in current across the biochemical sensing electrode and the reference electrode, and generating, at the processor, a quantitative biochemical response. Example implementations further include obtaining a biometric encryption key based on the biological surface, and encrypting the quantitative response based on a biometric encryption key. Example implementations further include contacting a fingerprint scanner to the biological surface, and obtaining a fingerprint pattern from the biological surface at the fingerprint scanner, where the biometric encryption key is based on the fingerprint pattern.

### BRIEF DESCRIPTION OF THE DRAWINGS

These and other aspects and features of the present implementations will become apparent to those ordinarily skilled in the art upon review of the following description of specific implementations in conjunction with the accompanying figures, wherein:

FIG. 1A illustrates an example device including a noninvasive biochemical sensor, in accordance with present implementations.

FIG. 1B illustrates an example device including a noninvasive biochemical sensor and a biometric fingerprint scanner, in accordance with present implementations.

FIG. 2A illustrates a plan view of an example hydrogel capsule and hydrogel layer integrable with an example biochemical sensor, in accordance with present implementations.

FIG. 2B illustrates a cross-sectional view of the example hydrogel capsule of FIG. 2A.

FIG. 3A illustrates a plan view of an example noninvasive biochemical sensor, in accordance with present implementations.

FIG. 3B illustrates a cross-sectional view of the example noninvasive biochemical sensor of FIG. 3A.

FIG. 4 illustrates an example electronic device including a noninvasive biochemical sensor, in accordance with present implementations.

FIG. 5A illustrates a first method of manufacturing a hydrogel layer and a hydrogel capsule, in accordance with present implementations.

FIG. 5B illustrates a second method of manufacturing a hydrogel layer and a hydrogel capsule, in accordance with present implementations.

FIG. 6A illustrates a first method of manufacturing a noninvasive biochemical sensor device, in accordance with present implementations.

FIG. 6B illustrates a second method of manufacturing a noninvasive biochemical sensor device, in accordance with present implementations.

FIG. 7 illustrates an example method of noninvasively sensing a biochemical and, optionally, biometrically securing biochemical sensor input, in accordance with present implementations.

FIG. 8 illustrates an example method of noninvasively sensing a biochemical and, optionally, biometrically securing biochemical sensor input, further to the example method of FIG. 7.

FIG. 9 illustrates an example method of noninvasively sensing a biochemical, in accordance with present implementations.

### DETAILED DESCRIPTION

The present implementations will now be described in detail with reference to the drawings, which are provided as illustrative examples of the implementations so as to enable those skilled in the art to practice the implementations and alternatives apparent to those skilled in the art. Notably, the figures and examples below are not meant to limit the scope of the present implementations to a single implementation, but other implementations are possible by way of interchange of some or all of the described or illustrated elements. Moreover, where certain elements of the present implementations can be partially or fully implemented using known components, only those portions of such known components that are necessary for an understanding of the present implementations will be described, and detailed descriptions of other portions of such known components will be omitted so as not to obscure the present implementations. Implementations described as being implemented in software should not be limited thereto, but can include implementations implemented in hardware, or combinations of software and hardware, and vice-versa, as will be apparent to those skilled in the art, unless otherwise specified herein. In the present specification, an implementation showing a singular component should not be considered limiting; rather, the present disclosure is intended to encompass other implementations including a plurality of the same component, and vice-versa, unless explicitly stated otherwise herein. Moreover, applicants do not intend for any term in the specification or claims to be ascribed an uncommon or special meaning unless explicitly set forth as such. Further, the present implementations encompass present and future known equivalents to the known components referred to herein by way of illustration.

Sweat-based biofluid biomarker sensing modality advantageously provides non-invasive access to molecular-level information, enabling insight into the body's dynamic chemistry. Stimulation-based methods are advantageous in certain environments and subjects. However, certain forms of stimulation can be effective in a subset of desired application scenarios. As one example exercise requires strenuous physical activity that may not be suitable for all potential users, including bedridden, neonatal, or elderly populations. As another example, heat applied to a user's body at a sensor site to stimulate sweat can lead to exhaustion and discomfort, which can contribute to a lowered rate of compliance where used in a device for patients. As another example, iontophoresis has numerous challenges related to the integration of device- and circuit-level stimulating components, increasing device complexity and cost. In accordance with present implementations, biofluids include but are not limited to human sweat.

Thus, it is advantageous to access sweat biomarkers through naturally occurring and background thermoregulatory perspiration, natural perspiration, or the like. In some implementations, natural perspiration-based sampling eliminates the need for active stimulation and provides a stable secretion rate within a time window of sampling. In some implementations, this stable secretion advantageously minimizes confounding effects of sweat rate variability and leads to more accurate analysis. In some implementations, a stable secretion rate is or is substantially 1 nL/min per sweat gland.

In some implementations, one or more of drugs, metabolites, and the like of target compounds can be extracted from sweat, advantageously yielding information about the individual's consumption of foreign, illicit, or like substances with the added potential to make inferences regarding the individual's general health state. In some implementations, a latent fingerprint image to uniquely identify an individual is constructible from one or more of spatial distribution of extracted analytes in obtained biofluid, and ridge and groove patterns of the sweat left on the surface. Thus, in-situ electrochemical techniques including a reliable medium to envelop and protect sensing electrodes as well as facilitate analyte transport to those electrodes for detection are advantageous to in vivo capture and analysis of biofluids including sweat.

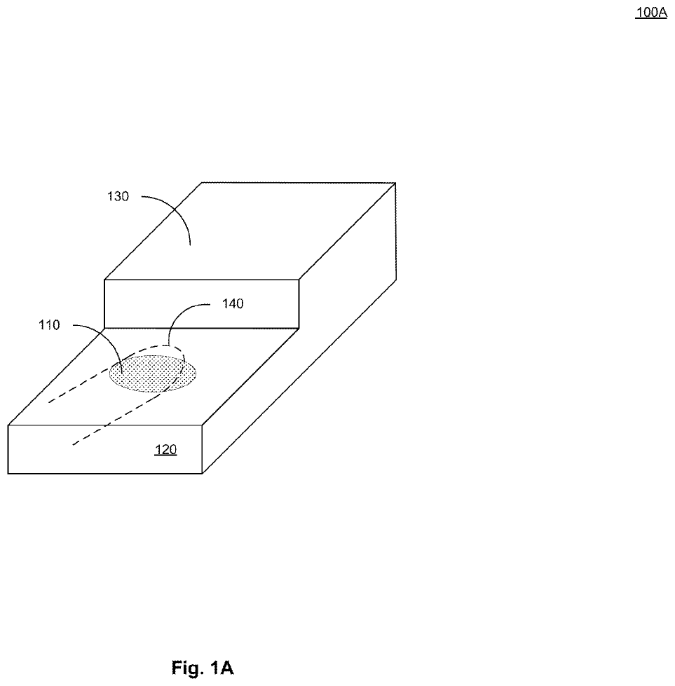

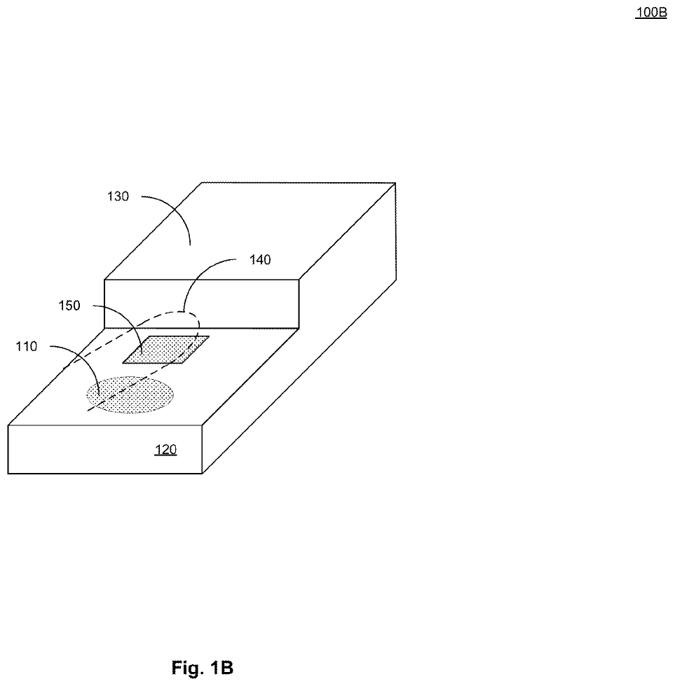

FIG. 1A illustrates an example device including a noninvasive biochemical sensor, in accordance with present implementations. As illustrated by way of example in FIG. 1A, example device 100A includes a biochemical sensor hydrogel 110, a housing 120, and an electronics region 130 of the housing 120. The biochemical sensor hydrogel 110 of the example device 100A is contactable with a biological surface 140.

The biochemical sensor hydrogel 110 is operable to absorb naturally secreted biofluid from a biological surface, and to transport the biofluid to an electrochemical sensor. In some implementations, a hydrogel is integrable into health monitoring devices. In some implementations, the biochemical sensor hydrogel 110 is operable to detect one or more pharmacokinetic and metabolic profiles of living subjects through contact with biological surfaces of those living subjects. In some implementations, the biochemical sensor hydrogel has a sampling time response of five minutes to collect a sufficient amount of naturally secreted biofluid to support an accurate electrochemical response to a present biofluid. In some implementations, sweat secretion and capture is inhibited by surface tension. In some implementations, a surface tension-induced Laplace pressure opposes sweat secretion, where the surface tension corresponds to that of a sweat droplet with a finite radius of curvature forming on the surface of the skin. In some implementations, the biochemical sensor hydrogel 110 reduces or eliminates the PL pressure barrier. In some implementations, by creating a continuous hydrophilic fluidic path connecting a sweat gland of a biological surface to an electrochemical biofluid sensor, the biochemical sensor hydrogel 110 reduces PL to zero. In addition, due to its aqueous nature, the hydrogel interface of the biochemical sensor hydrogel 110 also facilitates diffusion of analytes from sweat to the biochemical sensor hydrogel 110.

In some implementations, the biochemical sensor hydrogel is formed as a thin hydrogel micropatch (THMP). In some implementations, the THMP is formed with a planar structure. In some implementations, the THMP is approximately 340 μm-thick. In some implementations, the THMP has a thickness ranging among or between 340 μm, 510 μm, and 680 μm. In some implementations, the biochemical sensor hydrogel 110 formed as a THMP exhibits a physical relationship between thickness and electrochemical response time. In some implementations, a thinner THMP exhibits a shorter response time.

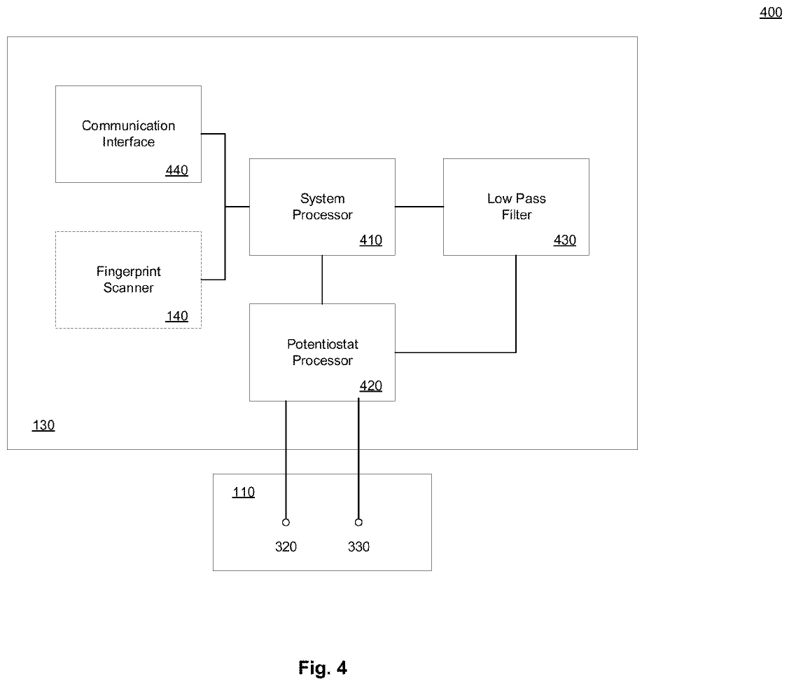

The housing 120 contains or the like one or more sensors, electrical devices, electronic devices, mechanical structures, and the like. In some implementations, the housing 120 includes a plastic material, a polymer material, electrically insulating material, waterproof material, water resistant material, or the like. The electronics portion 130 of the housing 120 houses one or more electronic components. In some implementations, the electronics portion houses at least one component of FIG. 4 it is to be understood that the housing 120 and the electronics component 130 thereof are not limited to the absolute or relative configuration of FIGS. 1A and 1B in accordance with present implementations.

The biological surface 140 is or includes a surface of living tissue, biological matter, or the like. In some implementations, the biological surface 250 includes partially or fully exposed skin or the like of a human, animal, plant, or the like. In some implementations, the biological surface secretes or is capable of secreting one or more fluids having one or more biochemicals therein. In some implementations, biochemical include, but are not limited to, lactate, caffeine, and the like. In some implementations, a biological surface is a finger, fingertip, or the like. In some implementations, sweat is collected from at least one finger, fingertips, and the like. In some implementations, it is advantageous to obtain sweat from fingertips due to relatively high density of eccrine sweat glands therein and blood capillaries in close proximity to fingertip sweat glands. In some implementations, biological surfaces can include on-body skin sites at any location thereon, and are not limited to finger, fingertip, or like surfaces.

FIG. 1B illustrates an example device including a noninvasive biochemical sensor and a biometric fingerprint scanner, in accordance with present implementations. As illustrated by way of example in FIG. 1B, example device 100B includes the biochemical sensor hydrogel 110, the housing 120, the electronics region 130 of the housing 120, and a fingerprint scanner device 150. The one or more of the biochemical sensor hydrogel 110 and the fingerprint scanner device 150 of the example device 100B are contactable with the biological surface 140.

The fingerprint scanner device 150 is operable to capture surface topography characteristics associated with the biological surface, including a fingerprint of a finger. In some implementations, an example system captures fingerprint identifiers of individuals. In some implementations, the example system takes at least one biometric identifier reading with shared terminals at different time points. In some implementations, an example device captures both biochemical and identification information by a one-touch biochemical sensing and user identification system. Thus, in some implementations, user identification along with biomarker sensing eliminates an extra step for the user intervention, allowing for the convenient use of terminals for biomarker sampling. In some implementations, a captured fingerprint is or can be used to generate a unique key associated with an individual for biochemical data encryption and decryption. Thus, in some implementations, only the subject and authenticated people have access to biochemical response data detected and generated by the example system in accordance with present implementations. In some implementations, combining biometric security through fingerprint recognition and biochemical sensing into a single device or system is advantageous to ensure security of obtained personal health data. In some implementations, an example device in accordance with present implementations, is embedded within an Internet-of-Things (IoT) infrastructure for community-based health monitoring.

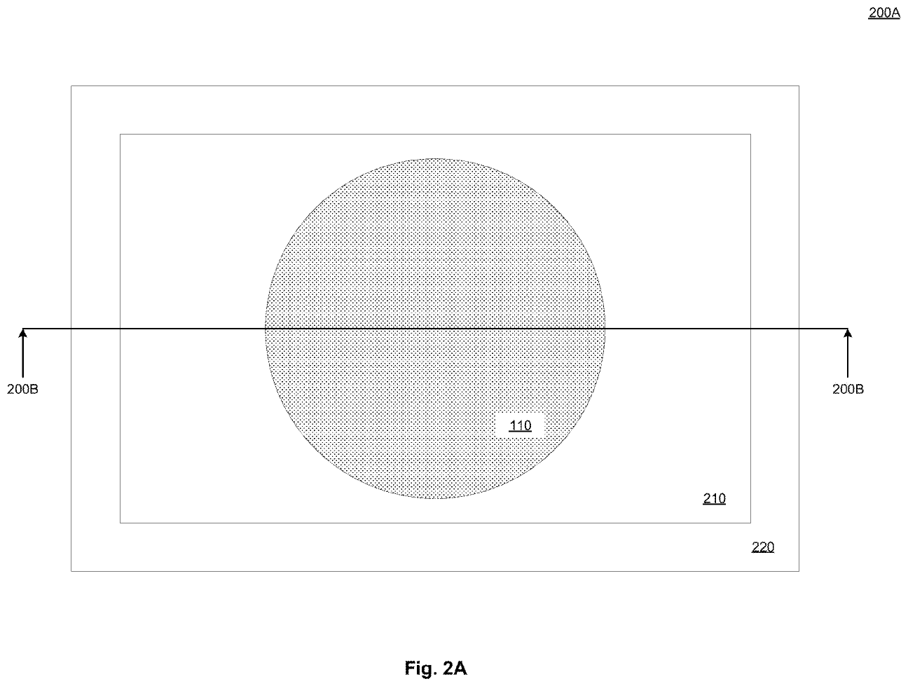

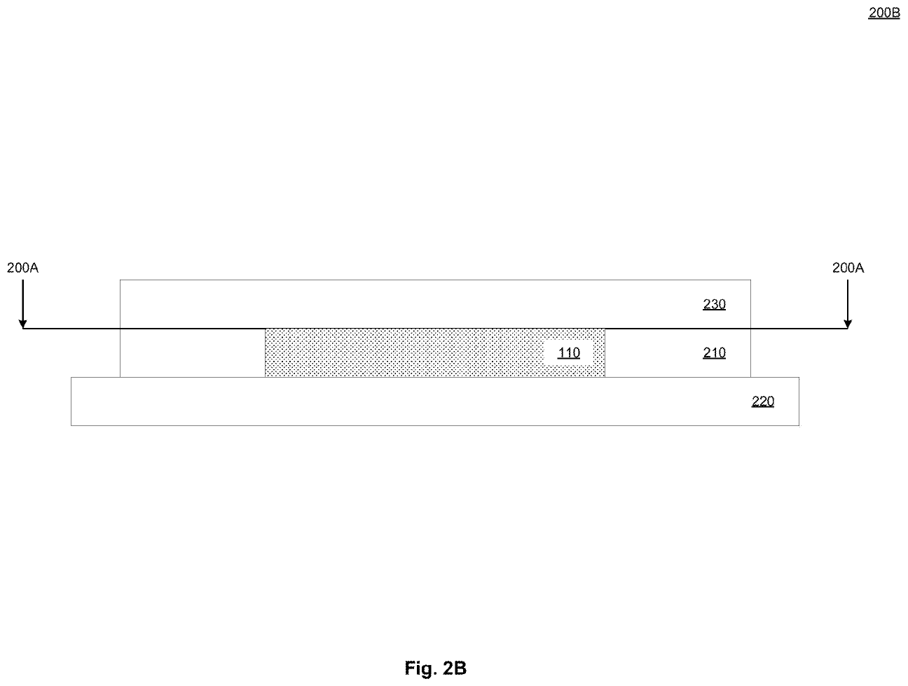

FIG. 2A illustrates a plan view of an example hydrogel capsule and hydrogel layer integrable with an example biochemical sensor, in accordance with present implementations. As illustrated by way of example in FIG. 2A, example hydrogel capsule and hydrogel layer 200A include the biochemical sensor hydrogel 110, a hydrogel capsule substrate 210, and a microfluidic layer 220. The hydrogel capsule substrate 210 is operable to form a storage-quality surface on which at least one biochemical sensor hydrogel 110 is formed or formable. In some implementations, the hydrogel capsule substrate is glass or the like.

The microfluidic layer 220 includes at least one cavity in at least one structure operable to capture fluid. In some implementations, the cavity of the microfluidic layer 220 includes at least one opening in a planar structure thereof. In some implementations, the microfluidic layer 220 is or includes a flexible planar structure. As one example, the microfluidic structure can be a flexible plastic film. As another example, the microfluidic structure can be a flexible plastic adhesive tape. In some implementations, the microfluidic layer includes a plurality of layers arranged in a stack. As one example, the microfluidic layer 220 includes a plurality of stacked adhesive tape films of a predetermined thickness. In some implementations, a predetermined thickness of a single adhesive tape film is 170 μm. It is to be understood that the microfluidic layer 220 can include an arbitrary number of films to achieve a particular desired height of the microfluidic layer.

FIG. 2B illustrates a cross-sectional view of the example hydrogel capsule of FIG. 2A. As illustrated by way of example in FIG. 2B, example hydrogel capsule and hydrogel layer 200B include the biochemical sensor hydrogel 110, the hydrogel capsule substrate 210, the microfluidic layer 220, and a capping layer 230.

The capping layer 230 includes a planar structure bonded to the microfluidic layer 220. In some implementations, the capping layer 230 is a flexible plastic layer. In some implementations, the capping layer 230 is or includes a polyethylene terephthalate (PET) film. In some implementations, the capping layer 230 includes one or more openings. In some implementations, the opening includes at least one opening from one planar surface of the capping layer 230 to another planar surface. In some implementations, the opening is or includes one or more vias or the like.

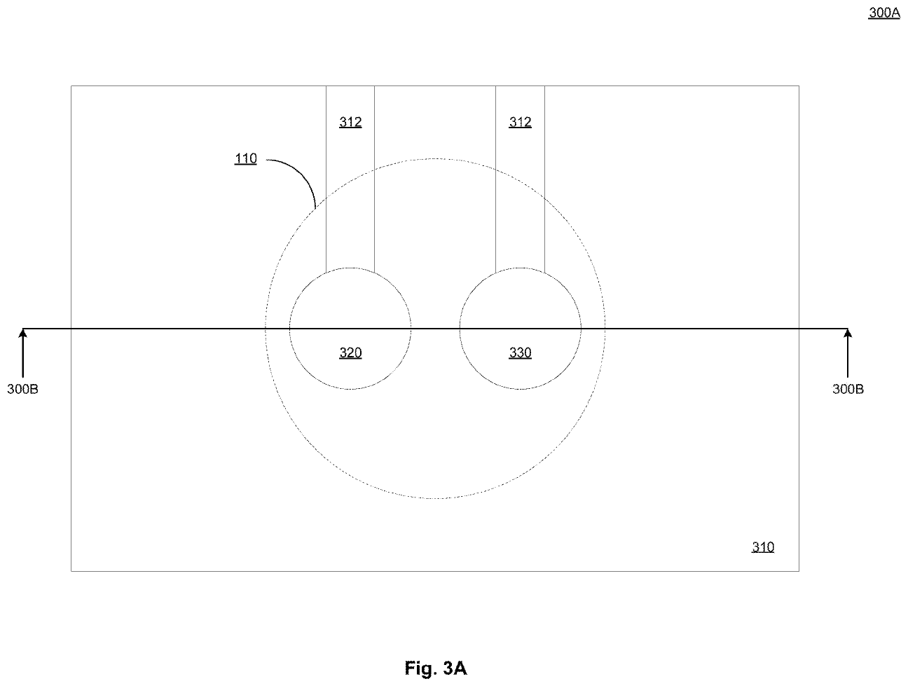

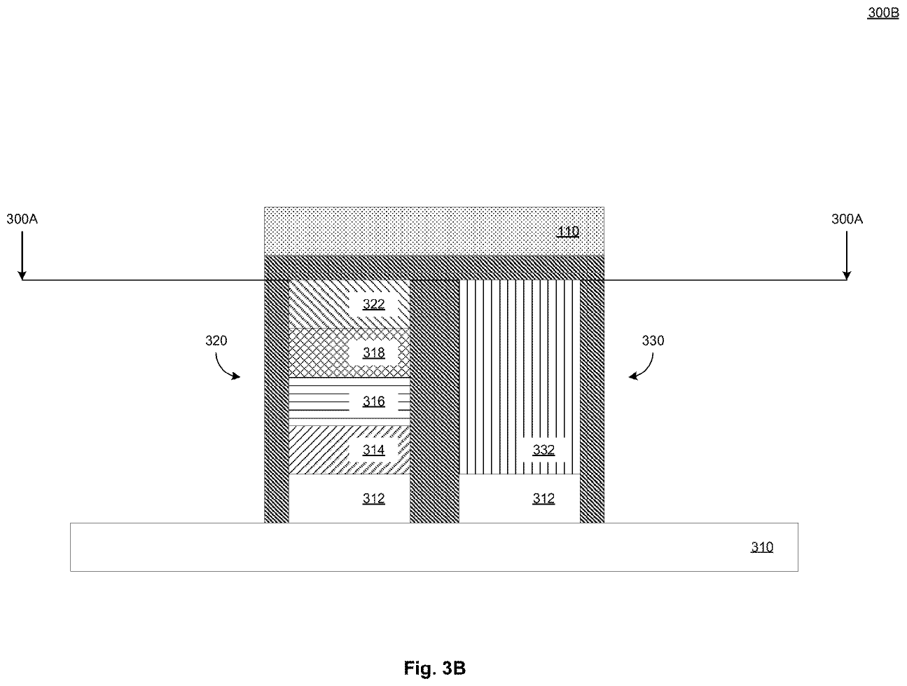

FIG. 3A illustrates a plan view of an example noninvasive biochemical sensor, in accordance with present implementations. As illustrated by way of example in FIG. 3A, example noninvasive biochemical sensor 300A includes the biochemical sensor hydrogel 110, an electrode substrate 310, electrode leads 312, a biochemical sensor electrode 320, and a reference electrode 330. In some implementations, the example biochemical sensor includes a THMP affixed upon a flexible PET substrate patterned with functionalized Au electrodes.

The electrode substrate 310 includes a planar surface having at least one electrode formed thereon. In some implementations, the electrode layer 210 has a first planar surface having one or more electrodes patterned, deposited, or the like, thereon. In some implementations, the electrode layer 210 is or includes a polyethylene terephthalate (PET) film. In some implementations, the electrode layer 210 is operatively coupled to one or more electrical, electronic, or like components housed at least partially within the housing 110.

The electrode leads 312 include at least one metallic portion forming at least part of at least one electrode terminal, biochemically-sensitive electrode terminal, or a combination thereof. In some implementations, the electrode leads 312 include electrode material disposed on a first planar surface of the electrode substrate 310 in one or more electrically isolated, electrically disconnected, or like configurations. In some implementations, the electrode leads 312 or includes one or more noble metal electrode films. As one example, the base leads 312 can include gold (Au) or the like, or a combination thereof.

The biochemical sensor electrode 320 is operable to electrochemically respond to contact with one or more target analytes received from the biochemical sensor hydrogel 110. In some implementations, the biochemical sensor electrode is in direct or indirect contact with the biochemical sensor hydrogel 110 formed as the THMP. In some implementations, the biochemical sensor electrode 320 includes a plurality of conductive or semiconductor materials. In some implementations, one or more conductive or semiconductor materials sequentially deposited to form the biochemical sensor electrode 320 mix, combine, or the like into a single layer or an interface between two layers. The reference electrode 330 is operable to electrically conduct a response current across the biochemical sensor hydrogel 110 together with the biochemical sensor electrode 320. In some implementations, the reference electrode is electrically responsive and electrochemically nonresponsive.

FIG. 3B illustrates a cross-sectional view of the example noninvasive biochemical sensor of FIG. 3A. As illustrated by way of example in FIG. 3A, example noninvasive biochemical sensor 300A includes the biochemical sensor hydrogel 110, the electrode substrate 310, the electrode leads 312, the biochemical sensor electrode 320, the reference electrode 330, and an encapsulating layer 340. In some implementations, the biochemical sensor electrode 320 includes one or more of a carbon nanotube layer 322, a platinum layer 324, a poly-m-phenylenediamine (PPD) layer 326, and a lactate oxide layer 328. In some implementations, the reference electrode 330 includes a silver chloride layer 332.

The carbon nanotube layer 322 is disposed over one of the electrode leads 312. In some implementations, the carbon nanotube layer 322 is or includes a multi-walled carbon nanotube (MWCNT) layer. The platinum layer 324 is disposed over the carbon nanotube layer 312. In some implementations, the platinum layer includes platinum nanoparticles (PtNP). In some implementations, a MWCNT/PtNP layer, advantageously enhances sensor sensitivity by the nanostructure's high catalytic activity and large surface area. In some implementations, the MWCNT and PtNP is formed into a single layer including carbon nanotube layer 312 and the platinum layer 322.

The PPD layer 326 is disposed over the platinum layer 326. In some implementations, the PPD layer advantageously mitigates confounding effect of electroactive species present in sampled biofluid. In some implementations, confounding effects include distortion of current response leading to an inaccurate inference of quantity of targe analyte in biofluid absorbed by the biochemical sensor hydrogel 110. As one example, inteferents include but are not limited to uric acid (UA) and ascorbic acid (AA). The lactate oxide layer (LOx) 328 is disposed over the PPD layer 326. In some implementations, the LOx layer advantageously generates hydrogen peroxide in proportion to concentration of lactate, other target analyte, or the like, absorbed in the biochemical sensor hydrogel 110. The encapsulating layer 340 is disposed over the LOx layer 328. In some implementations, the encapsulating layer 340 is or includes a PVC layer contactably interfacing with the biochemical sensor hydrogel 110. In some implementations, the encapsulating layer 340 is a diffusion-limiting membrane that enhances the biochemical sensor system's dynamic range in accordance with present implementations.

FIG. 4 illustrates an example electronic device including a noninvasive biochemical sensor, in accordance with present implementations. As illustrated by way of example in FIG. 4, example electronic device 400 includes the biochemical sensor hydrogel 110 and the electronics portion 130 of the housing 120. In some implementations, the example electronic device 400 further includes a system processor, a potentiostat processor, a low pass filter 430, and a communication interface 440. In some implementations, the example electronic device 400 further includes the fingerprint scanner 140. In some implementations, the example electronic device 400 further includes the biochemical sensor electrode 320 and the reference electrode 330. In some implementations, a sensor current path 450 lies through the biochemical sensor hydrogel 110 and between the biochemical sensor electrode 320 and the reference electrode 330.

The system processor 410 is operable to execute one or more instructions associated with input responsive to contact between the biochemical sensor hydrogel 110 and a biological surface. In some implementations, the system processor 410 is an electronic processor, an integrated circuit, or the like including one or more of digital logic, analog logic, digital sensors, analog sensors, communication buses, volatile memory, nonvolatile memory, and the like. In some implementations, the system processor 310 includes but is not limited to, at least one microcontroller unit (MCU), microprocessor unit (MPU), central processing unit (CPU), graphics processing unit (GPU), physics processing unit (PPU), embedded controller (EC), or the like. In some implementations, the system processor 410 includes a memory operable to store or storing one or more instructions for operating components of the system processor 410 and operating components operably coupled to the system processor 410. In some implementations, the one or more instructions include at least one of firmware, software, hardware, operating systems, embedded operating systems, and the like. It is to be understood that the system processor 410 or the example electronic device 400 generally can include at least one communication bus controller to effect communication between the system processor 410 and the other elements of the device 400. In some implementations, the system processor 410 is an ultra-low-power microcontroller unit. In some implementations, the system processor 410 is operatively coupled to at least one of a battery unit and a power source.

The potentiostat processor 420 includes one or more logical or electronic devices including but not limited to integrated circuits, logic gates, flip flops, gate arrays, programmable gate arrays, and the like. In some implementations, the potentiostat processor 420 applies +0.5 V across the biochemical sensor electrode 320 and the reference electrode 330. In some implementations, the potentiostat processor is operable to programmably change applied voltage to a value greater than or less than +0.5 V. In some implementations, the potentiostat processor 420 includes or is operatively coupled to a transimpedance amplifier. In some implementations, the potentiostat 420 converts an obtained current signal to an analog or digital voltage value by the transimpedance amplifier.

The low pass filter 430 is operable to minimize noise in the electrical response current received from one or more of the biosensor electrode terminals. The corresponding output voltage was filtered by a fifth-order low-pass filter module.

The communication interface 440 is operable to communicatively couple at least the system processor 410 to at least one external device. In some implementations, the communication interface 440 includes one or more wired interface devices, channels, and the like. In some implementations, the communication interface 440 includes, is operably coupled to, or is operably couplable to an I2C, UART, or like communication interface by one or more external devices, systems, or the like. In some implementations, the communication interface 440 includes a network or an Internet communication interface or is operably couplable to an Internet communication interface by one or more external devices, systems, or the like. In some implementations, the communication interface 440 is or includes a wireless transceiver operable to wirelessly and bilaterally communicate user commands and the sensor output current. In some implementations, the communication interface 440 is or includes a Bluetooth™ transceiver. In some implementations, the communication interface 440 communication in real-time with an external device. In some implementations, an external device includes a custom-developed smartphone application compatible with the output of the potentiostat processor 420.

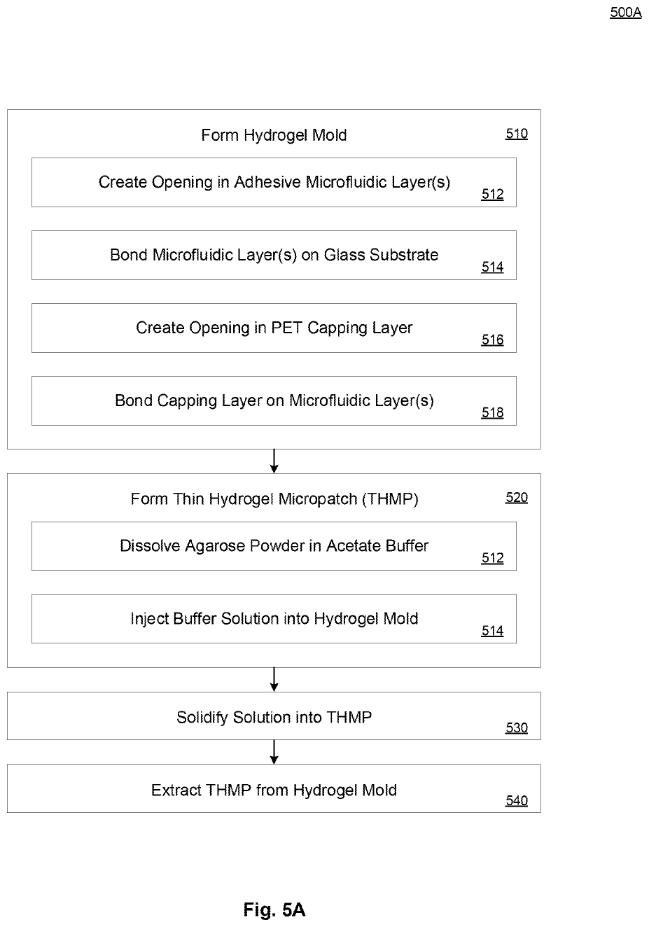

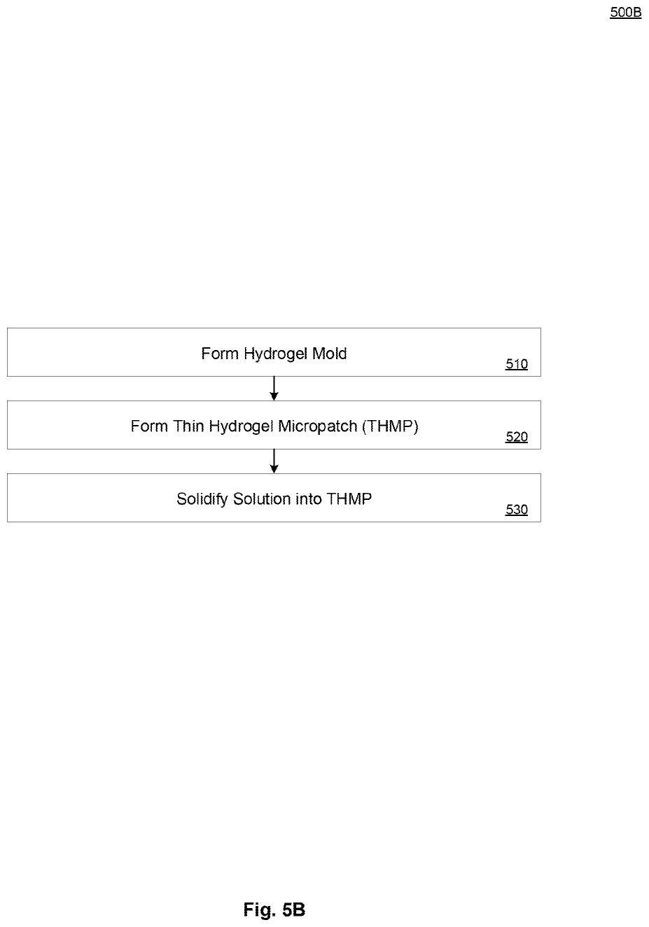

FIG. 5A illustrates a first method of manufacturing a hydrogel layer and a hydrogel capsule, in accordance with present implementations. In some implementations, at least one portion of the example devices 100A and 100B is formed by method 500A according to present implementations. In some implementations, the method 500A begins at step 510.

At step 510, the example system forms a hydrogel mold. In some implementations, a THMP is prepared by a layered mold assembly of a glass slide substrate, a middle layer consisting of one or more strips of double-sided tape, and a PET capping layer. In some implementations, step 510 includes at least one of steps 512, 514, 516 and 518. At step 512, the example system creates an opening in at least one microfluidic layer 220. In some implementations, the example system creates an opening by laser-cutting the microfluidic layer 220. In some implementations, the microfluidic layer is cut to at least partially surround a chamber corresponding to a size and shape of a THMP. In some implementations, the opening is substantially circular. In some implementations, the opening has a diameter of approximately 6 mm. In some implementations, the microfluidic layer is or includes at least one adhesive material disposed on at least one planar surface thereof. As one example, the microfluidic layer can be a single-sided or double-sided adhesive tape. In some implementations, the microfluidic layer is or includes multiple films, tapes, layers, or the like arranged in a stack or the like. In some implementations, thickness of the biochemical sensor hydrogel 110 is controllably adjusted by varying the number of double-sided tape strips used to create the wall of the microfluidic chamber. At step 514, the example system bonds the microfluidic layer on a substrate. In some implementations, the substrate is or includes a glass or like material. At step 516, the example system creates at least one opening in a capping layer. In some implementations, the capping layer is a PET layer. In some implementations, the example system creates an opening by laser-cutting the microfluidic layer 220. In some implementations, the opening is substantially circular. In some implementations, the opening has a diameter of approximately 1 mm. In some implementations, the opening is or includes an inlet or outlet to facilitate injection of hydrogel or like solution into the chamber through the PET layer.

At step 518, the example system bonds the capping layer on the microfluidic layer. In some implementations, the example system thus form a cavity bordered by the capping layer above, the substrate below, and the microfluidic layers to each side. The method 500A then continues to step 520.

At step 520, the example system forms a thin hydrogel micropatch (THMP). In some implementations, step 520 includes at least one of steps 522 and 524. At step 522, the example system dissolves agarose powder in a buffer liquid. In some implementations, the buffer liquid is or includes an acetate buffer. In some implementations, the example system combines agarose and sodium acetate buffer solution. In some implementations, the agarose solution has a concentration of approximately 3 M, and a pH of 5.2±0.1. In some implementations, the hydrogel solution contains 2% agarose. In some implementations, agarose powder is dissolved by 0.01 M acetate buffer. In some implementations, the hydrogel solution is diluted from 3 M stock solution, at a pH of 5, to approximate characteristics of and compatibility with absorption of sweat. In some implementations, the resulting mixture is placed in an 80° C. water bath. It is to be understood that the agarose powder can be a mixture including other materials. It is to be further understood that powders, solids, or the like, with one or more characteristics corresponding to agarose can be incorporated with or replace agarose powder. At step 524, the example system injects a buffer solution into the hydrogel mold. In some implementations, the buffer solution includes the dissolved agarose powder and the acetate buffer, or substitutes in accordance with present implementations. The method 500A then continues to step 530.

At step 530, the example system solidifies the buffer solution into the THMP. In some implementations, after agarose is completely dissolved, the solution is immediately injected into the pre-assembled hydrogel mold. In some implementations, the hydrogel mold is heated to 80° C. The method 500A then continues to step 540.

At step 540, the example system extracts the THMP from the hydrogel mold. In some implementations, response characteristics of the extracted THMP can be obtained. In some implementations, calibration of the THMP is performed with THMPs pre-soaked with lactate solutions. In some implementations, lactate concentration levels in calibration are between 0 to 2 mM). In some implementations, the THMP-based lactate sensing module is challenged against a diverse panel of interferents at physiologically-relevant concentration levels. In some implementations, interferents further include electrolytes, electroactive species, small molecules, and the like. In some implementations, the example system presents a minimal normalized response to non-target analytes, demonstrating a high-level of selectivity. For example, a normalized response of 100% to 1 mM of lactate, corresponds to a normalized response of 10% to 100 uM of glucose, a normalized response of 20% to 10 mM of sodium ion, a normalized response of 10% to 2.4 mM of potassium ion, a normalized response of 20% to 50 uM of UA, and a normalized response of 15% to 10 uM of AA. In some implementations, the THMP is stored within the hydrogel mold for a period of time before removal and later use with a biochemical sensor device. Consider-ing the disposable nature of the THMP within the devised sensing system, the longtime storage of the THMP is advantageous. Thus, in some implementations, the hydrogel mold itself is a hydrophobic, plastic-based package. In some implementations, the method 500A ends at step 540.

FIG. 5B illustrates a second method of manufacturing a hydrogel layer and a hydrogel capsule, in accordance with present implementations. In some implementations, at least one portion of the example devices 100A and 100B is formed by method 500B according to present implementations. In some implementations, one or more of steps 510, 520 and 530 of the method 500B respectively correspond to at least a portion of steps 510, 520 and 530 of the method 500A.

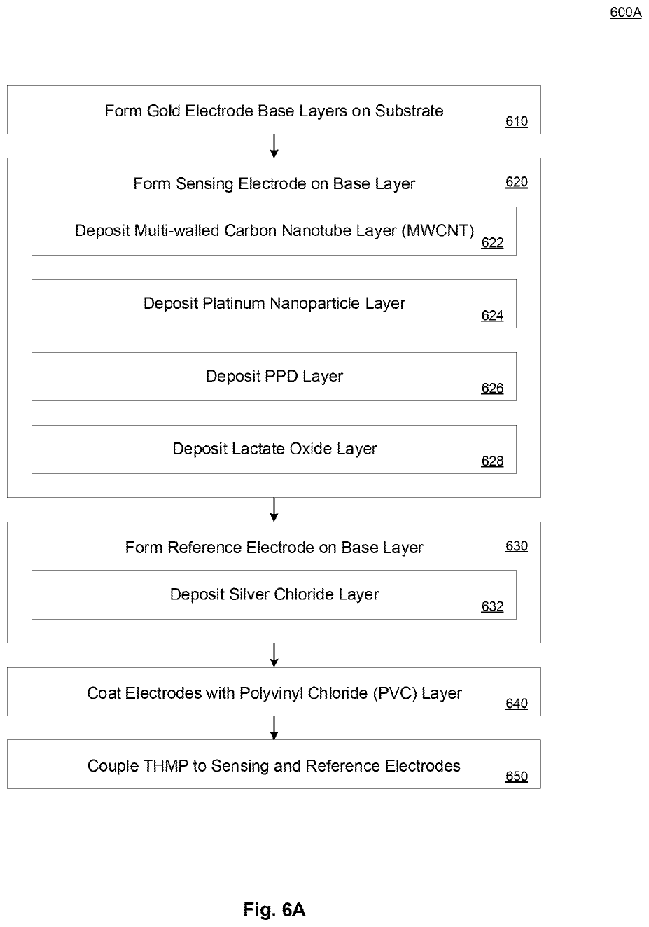

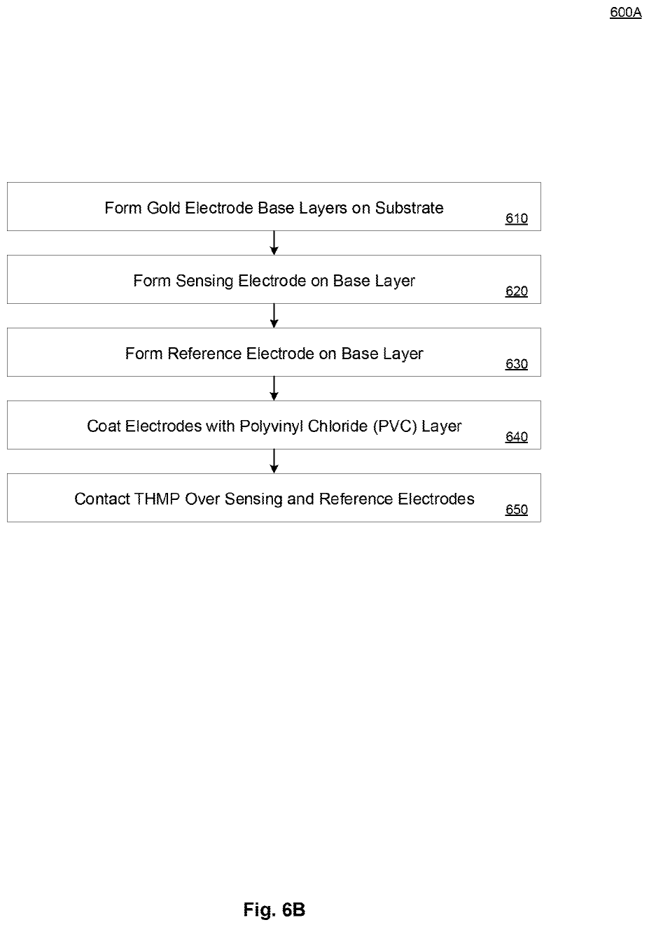

FIG. 6A illustrates a first method of manufacturing a noninvasive biochemical sensor device, in accordance with present implementations. In some implementations, at least one of the example devices 100A and 100B performs method 600A according to present implementations. In some implementations, the method 600A begins at step 610.

At step 610, the example system forms at least one electrode base layer on a substrate. In some implementations, the example system forms a first electrode base layer corresponding to a biochemical sensor electrode and a second electrode base layer corresponding to a reference electrode. In some implementations, the electrode base layer is or includes gold or the like. In some implementations, the electrode base layer is or includes a pair of gold (Au) electrodes having at least one of a diameter of 2.4 mm, and a composition of chromium film over gold base layer. In some implementations, the gold base layer is 100 nm in height, and the chromium film is 20 nm in height. In some implementations, the substrate is or includes PET or the like. The method 600A then continues to step 620.

At step 620, the example system forms a biochemical sensor electrode on the electrode base layer. In some implementations, the example system forms the biochemical sensor electrode only on the first electrode base layer. In some implementations, step 620 includes at least one of steps 622, 624, 626 and 628. At step 622, the example system deposits a carbon nanotube layer on the electrode base layer. In some implementations, the carbon nanotube layer is or includes a multi-walled carbon nanotube (MWCNT) layer. In some implementations, 1.15 μL of MWCNT solution is drop cast onto the Au electrode and dried at ambient environment conditions. In some implementations, the MWCNT solution is 2 mg/mL in 5 wt. % of Nafion solution. At step 624, the example system deposits a platinum layer on the carbon nanotube layer. In some implementations, platinum nanoparticles (PtNP) are electrochemically deposited onto the MWCNT modified Au electrode. In some implementations, the PtNP deposition occurs by PtNP in a deposition solution of 2.5 mM H2PtC16 and 1.5 mM formic acid in DI water. In some implementations, PtNP deposition is performed by cyclic voltammetry At step 626, the example system deposits a PPD layer on the platinum layer. In some implementations, the PPD layer is electrochemically deposited onto the Au/MWCNT/PtNP electrode with m-phenylenediamine solution. In some implementations, the phenylenediamine solution is 5 mM in PBS. In some implementations, the PPD deposition occurs by applying 0.85 V (vs. Ag/AgCl) for 120 s. At step 628, the example system deposits a lactate oxide layer on the PPD layer. In some implementations, Tbovine serum albumin (BSA) is used as a stabilizer and glutaraldehyde is used as a crosslinker to immobilize LOx. In some implementations, 500 μL of BSA solution at 10 mg/mL in PBS is mixed with 4 μL of glutaraldehyde solution at 25 wt. % in a combined solution. Then, in some implementations, the combined solution is mixed with the lactate oxidase solution at 10 mg/mL in PBS at a 1:1 volume-to-volume ratio to form an enzyme mixture. In some implementations, 1.15 μL of the enzyme mixture is drop cast onto the Au/MWCNT/PtNP/PPD electrode and dried at room temperature. It is to be understood that one or more layers can be omitted from deposition, and a layer may be thus deposited on a corresponding underlying layer are disclosed herein. The method 600A then continues to step 630.

At step 630, the example system forms a reference electrode on the electrode base layer. In some implementations, the example system forms the reference electrode only on the second electrode base layer. In some implementations, step 630 includes step 632. At step 632, the example system deposits a silver chloride layer on the electrode base layer. In some implementations, the reference electrode is fabricated by drop casting Ag/AgCl ink directly on the Au electrode, and baking at 80° C. for 10 min. The method 600A then continues to step 640.

At step 640, the example system coats at least one of the biochemical sensor electrode and the reference electrode with a PVC layer. In some implementations, the sensor is dip coated with PVC solution at 3 wt. % in THF. The method 600A then continues to step 650. At step 650, the example system couples a THMP to at least one of the biochemical sensor electrode and the reference electrode. In some implementations, the example system contacts the THMP over or indirectly with at least one of the biochemical sensor electrode and the reference electrode. In some implementations, the method 600A ends at step 650.

FIG. 6B illustrates a second method of manufacturing a noninvasive biochemical sensor device, in accordance with present implementations. In some implementations, at least one of the example devices 100A and 100B performs method 600B according to present implementations. In some implementations, one or more of steps 610, 620, 630, 640 and 650 of the method 600B respectively correspond to at least a portion of steps 610, 620, 630, 640 and 650 of the method 600A.

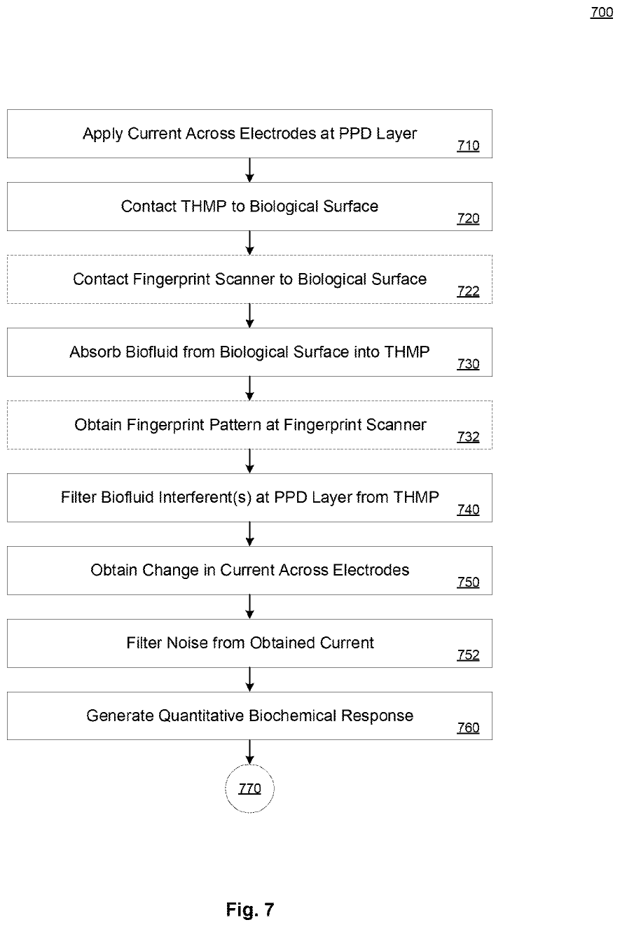

FIG. 7 illustrates an example method of noninvasively sensing a biochemical and, optionally, biometrically securing biochemical sensor input, in accordance with present implementations. In some implementations, at least one of the example devices 100A and 100B performs method 700 according to present implementations. In some implementations, the method 700 begins at step 710. At step 710, the example system applies current through a THMP by a biochemical sensor electrode and a reference electrode. The method 700 then continues to step 720. At step 720, the example system contacts the THMP to a biological surface. In some implementations, the example system contacts a top, upper, fingertip, or like, portion of a fingertip to the THMP. Alternatively, in some implementations, the example system contacts a bottom, lower, below fingertip, or like, portion of a finger to the THMP where a top, upper, fingertip, or like, portion of a fingertip contacts a fingertip scanner. The method 700 then continues to step 722. At step 722, the example system contacts a fingerprint scanner to the biological surface. In some implementations, the example system contacts a top, upper, fingertip, or like, portion of a fingerprint to the fingerprint scanner. It is to be understood that the method 700 can optionally include step 722. The method 700 then continues to step 730.

At step 730, the example system absorbs biofluid from the biological surface into the THMP. In some implementations, a THMP is placed on the skin for five minutes for sweat sampling. In some implementations, a poly-ethylene plastic film covers the device to avoid evaporation. In some implementations, lactate tracking on fingertips is performed by pressing the index finger-tip onto the THMP for five minutes, followed by taking an amperometric measurement upon the removal of the fingertip. In some implementations, THMP-based sampling of a fingertip is performed at 30-min intervals. in some implementations, measurements are taken before and after the consumption of target analyte. As one example, a beverage containing 150 mg of caffeine can be consumed. The method 700 then continues to step 732.

At step 732, the example system obtains at least one fingerprint pattern at the fingerprint scanner. It is to be understood that the method 700 can optionally include step 732. The method 700 then continues to step 740. At step 740, the example system filters at least one biofluid interferent. In some implementations, the PPD layer filters one or more biofluid interferents received in the biofluid from the THMP. The method 700 then continues to step 750.

At step 750, the example system obtains a change in current through the THMP. In some implementations, the example system generates a linear response of the developed sensing interface towards lactate over a range of 0 to 4 mM with a sensitivity of 1.88±0.24 μA/mM/cm2. In some implementations, an electrochemical sensor response is measured in relation to the hydrogel thickness to infer flux of the target molecules into the THMP. In some implementations, the electrochemical sensor response is proportional to the target concentration in the hydrogel. The method 700 then continues to step 752.

At step 752, the example system filters noise from the obtained current. The method 700 then continues to step 760.

At step 760, the example system generates a quantitative biochemical response. In some implementations, the method 700 then continues to step 770. Alternatively, in some implementations, the method 700 ends at step 760.

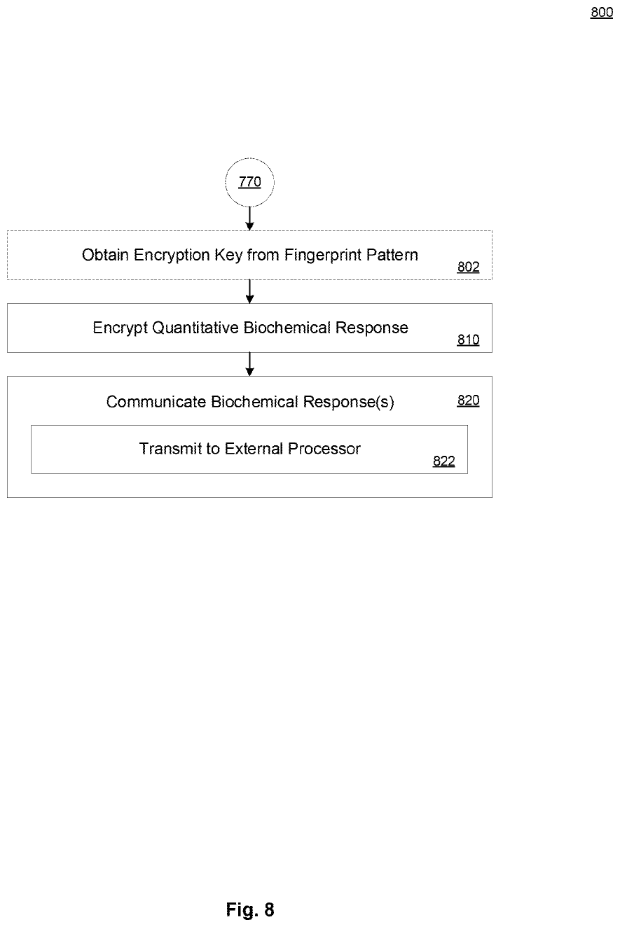

FIG. 8 illustrates an example method of noninvasively sensing a biochemical and, optionally, biometrically securing biochemical sensor input, further to the example method of FIG. 7. In some implementations, at least one of the example devices 100A and 100B performs method 800 according to present implementations. In some implementations, the method 800 begins at step 770. The method 800 then continues to step 802.

At step 802, the example system obtains an encryption key from the fingerprint pattern. In some implementations, the example system identifies a corresponding user by comparing an imaged fingerprint to a database of fingerprints. It is to be understood that the method 800 can optionally include step 802. The method 800 then continues to step 810.

At step 810, the example system encrypts the quantitative biochemical response. The method 800 then continues to step 820.

At step 820, the example system communicates the biochemical response. In some implementations, step 820 includes step 822. At step 822, the example system transmits the biochemical response to an external processor. In some implementations, the method 800 ends at step 820.

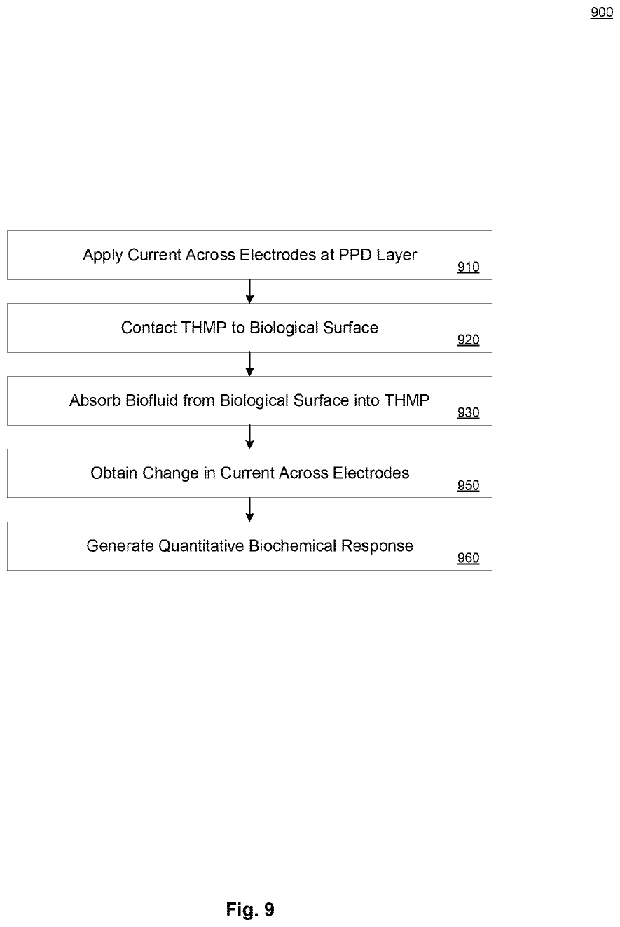

FIG. 9 illustrates an example method of noninvasively sensing a biochemical, in accordance with present implementations. In some implementations, at least one of the example devices 100A and 100B performs method 900 according to present implementations. In some implementations, one or more of steps 910, 920, 930, 950 and 960 of the method 900 respectively correspond to at least a portion of steps 710, 720, 730, 750 and 760 of the method 700.

The herein described subject matter sometimes illustrates different components contained within, or connected with, different other components. It is to be understood that such depicted architectures are illustrative, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components and/or wirelessly interactable and/or wirelessly interacting components and/or logically interacting and/or logically interactable components

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

It will be further understood by those within the art that if a specific number of an introduced claim recitation is intended, such an intent will be explicitly recited in the claim, and in the absence of such recitation, no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general, such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Further, unless otherwise noted, the use of the words “approximate,” “about,” “around,” “substantially,” etc., mean plus or minus ten percent.

The foregoing description of illustrative implementations has been presented for purposes of illustration and of description. It is not intended to be exhaustive or limiting with respect to the precise form disclosed, and modifications and variations are possible in light of the above teachings or may be acquired from practice of the disclosed implementations. It is intended that the scope of the invention be defined by the claims appended hereto and their equivalents.

1. A biochemical sensor device, comprising:
a substrate;
a first base electrode layer disposed on the substrate; and
a hydrogel layer disposed over the first base electrode layer.

2. The biochemical sensor device of claim 1, further comprising:
a carbon nanotube layer disposed on the first base electrode layer.

3. The biochemical sensor device of claim 2, further comprising:
a platinum layer disposed on the carbon nanotube layer.

4. The biochemical sensor device of claim 3, further comprising:
a poly-m-phenylenediamine (PPD) layer disposed on the platinum layer.

5. The biochemical sensor device of claim 4, further comprising:
a lithium oxide layer disposed on the PPD layer.

6. The biochemical sensor device of claim 1, further comprising:
a second base electrode layer disposed on the substrate.

7. The biochemical sensor device of claim 6, further comprising:
a silver chloride layer disposed on the second base electrode layer.

8. The biochemical sensor device of claim 6, further comprising:
a polyvinyl chloride (PVC) layer disposed over the first electrode base layer, over the second base electrode layer, and below the hydrogel layer.

9. The biochemical sensor device of claim 1, wherein the substrate comprises polyethylene terephthalate (PET).

10. The biochemical sensor device of claim 1, wherein the first base electrode comprises gold.

11. The biochemical sensor device of claim 1, wherein the second base electrode comprises gold.

12. An electronic sensor device, comprising:
a system processor; and
a sensor processor operatively coupled to the system processor;
a biochemical sensing electrode operatively coupled to the sensor processor; and
a hydrogel layer operatively coupled to the biochemical sensing electrode.

13. The electronic sensor device of claim 12, further comprising:
a reference electrode operatively coupled to the sensor processor, wherein the hydrogel layer is operatively coupled to the reference electrode.

14. The electronic sensor device of claim 12, further comprising:
a fingerprint scanner device operatively coupled to the system processor.

15. A method of manufacturing a biochemical sensor, comprising:
dissolving agarose powder in acetate buffer to form a buffer solution;
injecting the buffer solution into a hydrogel chamber; and
solidifying the buffer solution into a hydrogel layer.

16. The method of claim 15, further comprising:
bonding a first planar surface of a microfluidic layer to a substrate; and
bonding a capping layer to a second planar surface of the microfluidic layer to form the hydrogel chamber.

17. The method of claim 16, further comprising:
creating an opening in the microfluidic layer.

18. The method of claim 16, wherein the microfluidic layer includes adhesive material on at least one planar surface thereof.

19. The method of claim 16, further comprising:
creating an opening in the capping layer.

20. The method of claim 16, wherein the capping layer comprises polyethylene terephthalate (PET).

21. The method of claim 16, further comprising:
extracting the hydrogel layer from the hydrogel chamber.

22. The method of claim 16, further comprising:
storing the hydrogel layer in the hydrogel chamber.

23. A method of manufacturing a biochemical sensor, comprising:
depositing a first base electrode layer on a substrate;
depositing a carbon nanotube layer on the first base electrode; and
depositing a platinum layer on the carbon nanotube layer.

24. The method of claim 23, further comprising:
depositing a poly-m-phenylenediamine (PPD) layer on the platinum layer.

25. The method of claim 24, further comprising:
depositing a lactate oxide layer on the PPD layer.

26. The method of claim 23, further comprising:
depositing a second base electrode layer on the substrate.

27. The method of claim 26, further comprising:
depositing a silver chloride layer one the second base electrode.

28. The method of claim 23, further comprising:
forming a polyvinyl chloride (PVC) coating over the substrate.

29. The method of claim 26, further comprising:
coupling a hydrogel layer to the first base electrode layer and the second base electrode layer.

30. A method of sensing the presence and quantity of a biochemical, comprising:
applying a current across a biochemical sensing electrode and a reference electrode;
contacting a hydrogel layer to a biological surface;
absorbing a biofluid from the biological surface into the hydrogel layer;
obtaining, at a processor coupled to the biochemical sensing electrode and the reference electrode, a change in current across the biochemical sensing electrode and the reference electrode; and
generating, at the processor, a quantitative biochemical response.

31. The method of claim 30, further comprising:
filtering at least one interferent from the absorbed biofluid at a poly-m-phenylenediamine (PPD) layer disposed between the hydrogel layer and the biochemical sensing electrode and the reference electrode.

32. The method of claim 31, wherein the interference comprises at least one of glucose, sodium, potassium, uric acid, and ascorbic acid.

33. The method of claim 30, further comprising:
obtaining a biometric encryption key based on the biological surface; and
encrypting the quantitative response based on a biometric encryption key.

34. The method of claim 33, further comprising:
contacting a fingerprint scanner to the biological surface; and
obtaining a fingerprint pattern from the biological surface at the fingerprint scanner,
wherein the biometric encryption key is based on the fingerprint pattern.
